(19) United States
(12) Patent Application Publication
(10) Pub. No.: US 2022/0031668 A1
(43) Pub. Date: Feb. 3, 2022

(54) METHODS OF INHIBITING PROCOLLAGEN-LYSINE, 2-OXOGLUTARATE 5-DIOXYGENASE 2

(71) Applicant: University of Kentucky Research Foundation, Lexington, KY (US)

(72) Inventors: Ren Xu, Lexington, KY (US); Shike Wang, Lexington, KY (US)

(21) Appl. No.: 17/386,339

(22) Filed: Jul. 27, 2021

Related U.S. Application Data
(60) Provisional application No. 63/058,161, filed on Jul. 29, 2020.

(51) Int. Cl. A61K 31/427; A61K 31/7068; A61K 31/4439; A61K 31/205; A61K 38/13; A61K 31/519; A61K 31/137; A61K 31/192; A61K 31/451; A61K 31/16; A61K 31/4965; A61K 31/55; A61P 35/00

(52) U.S. Cl. CPC A61K 31/427; A61K 31/7068; A61K 31/4439; A61K 31/205; A61K 38/13; A61K 31/519; A61P 35/00; A61K 31/192; A61K 31/451; A61K 31/16; A61K 31/4965; A61K 31/55; A61K 31/137

(57) ABSTRACT

A method of inhibiting expression or activity of procollagen-lysine, 2-oxoglutarate 5-dioxygenases 2 (PLOD2) in a cell involves contacting the cell with or introducing into the cell an effective amount of a compound selected from the group consisting of: amiloride, azelastine, bazedoxifene acetate, BIBW2992, DL-carnitine, L-carnitine, cyclosporin A, dopamine, gallic acid, gemcitabine, loperamide, manidipine, marimastat, methacycline, mubritinib, P1015, P1025, P1029, palbociclib, pexidartinib, rosiglitazone, tazemetostat, tebipenem pivoxil, teneligliptin, trospium chloride, and pharmaceutically-acceptable salts thereof.

### RELATED APPLICATIONS

This application claims priority from U.S. Provisional Application Ser. No. 63/058,161 filed Jul. 29, 2020, the entire disclosure of which is incorporated herein by this reference.

### TECHNICAL FIELD

The presently-disclosed subject matter generally relates to methods of inhibiting procollagen-lysine, 2-oxoglutarate 5-dioxygenases 2 (PLOD2) expression and/or activity. In particular, certain embodiments of the presently-disclosed subject matter relate to methods of inhibiting cancer cell migration using a PLOD2 inhibitor.

### INTRODUCTION

Collagen is one of the major components of extracellular matrix. The collagen-cell interaction induces biochemical and biophysical signals, which is essentially for normal tissue function and cancer progression (Egeblad et al., 2010; Xiong and Xu, 2016). Collagen is the most abundant protein in our body, and presents in both normal tissues and cancer.

Collagen regulates tumor progression by modulating cancer cell migration, invasion (Xiong et al., 2014), proliferation (Pollard, 2004), survival (Cheon et al., 2014) and metastasis (Oudin et al., 2016; Sun et al., 2016).

All collagen is composed of a triple helix, and the most common motif of the triple helix sequence is Gly-X-Y (X and Y represent proline or hydroxyproline) (Albaugh et al., 2017). Collagen cross-linking and deposition depend on lysyl hydroxylation, which is catalyzed by procollagen-lysine, 2-oxoglutarate 5-dioxygenases (PLOD). (Qi 2018; Valtavaara 1998). The hydroxylation of lysyl residues is one of the critical steps of collagens biosynthesis. It usually occurs in the Y position of the repeating Gly-X-Y motif (Barnes 1974; Valtavaara 1998). PLOD-dependent collagen crosslinking stabilizes newly formed collagen fibers and enhances the stiffness of the matrix.

Three isoforms of PLOD have been identified—PLOD1, PLOD2, and PLOD3, which are sometimes referred to as lysyl hydroxylase-1, -2, and -3 (Hausmann, 1967; Rhoads and Udenfriend, 1968; Kivirikko Ki, 1998; Rautavuoma 2004). PLOD1 and PLOD3 hydroxylate lysyl residues in the collagen triple helix; however, only PLOD2 has been identified to hydroxylate lysyl residues in the telopeptides of collagen (Gilkes 2013; Takaluoma 2007), thereby driving stable collagen cross-linking (Uzawa 1999).

The overexpression of PLODs have been found to contribute to many collagen-related diseases (Gjaltema 2017; Qi 2018; Li 2020). High expression of PLOD2 increases tumor stiffness, promotes cancer metastasis, contributes to the development of drug resistance, and predicts the poor clinical outcome in breast cancer, lung cancer, sarcoma, and other tumor types (Gilkes 2013b; Chen 2015; Eisinger-Mathason 2013; Monferrer 2019; Guo 2018; Chen 2016; Kiyozumi 2018; Okumura 2018). Overexpression of PLOD2 has also been found to promote invasion and migration of tumor cells. (Xu 2017).

Accordingly, PLOD2 appears to be a potential target for cancer therapy; however, there have not been any PLOD2 inhibitors introduced for use in clinical therapy. Accordingly, there is a need in the art for effective PLOD2 inhibitors and methods of inhibiting PLOD2.

### SUMMARY

The presently-disclosed subject matter meets some or all of the above-identified needs, as will become evident to those of ordinary skill in the art after a study of information provided in this document.

This Summary describes several embodiments of the presently-disclosed subject matter, and in many cases lists variations and permutations of these embodiments. This Summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently-disclosed subject matter, whether listed in this Summary or not. To avoid excessive repetition, this Summary does not list or suggest all possible combinations of such features.

The presently-disclosed subject matter includes a method of inhibiting the expression or activity of procollagen-lysine, 2-oxoglutarate 5-dioxygenases 2 (PLOD2) in a cell. In some embodiments, the method involves contacting the cell with or introducing into the cell an effective amount of a compound, wherein the compound is selected from the group consisting of: amiloride, azelastine, bazedoxifene acetate, BIBW2992, DL-carnitine, L-carnitine, cyclosporin A, dopamine, gallic acid, gemcitabine, loperamide, manidipine, marimastat, methacycline, mubritinib, P1015, P1025, P1029, palbociclib, pexidartinib, rosiglitazone, tazemetostat, tebipenem pivoxil, teneligliptin, trospium chloride, and pharmaceutically-acceptable salts thereof, wherein the contacting or introducing results in inhibition of expression or activity of the PLOD2 in the cell.

In some embodiments of the method of inhibiting expression or activity of PLOD2, the compound is selected from the group consisting of: amiloride, azelastine, DL-carnitine, L-carnitine, gallic acid, loperamide, tebipenem pivoxil, and pharmaceutically-acceptable salts thereof. In some embodiments the compound is amiloride or a pharmaceutically-acceptable salt thereof. In some embodiments the compound is azelastine or a pharmaceutically-acceptable salt thereof. In some embodiments the compound is loperamide or a pharmaceutically-acceptable salt thereof.

In some embodiments of the method of inhibiting expression or activity of PLOD2, the compound is provided in a pharmaceutical composition further comprising a pharmaceutically-acceptable carrier.

In some embodiments of the method of inhibiting expression or activity of PLOD2, the cell is a cancer cell. In some embodiments, the cancer cell is a breast cancer cell. In some embodiments, the cancer cell is a lung cancer cell. In some embodiments, the cancer cell is a colorectal cancer cell.

In some embodiments of the method of inhibiting expression or activity of PLOD2, the cell is in a subject. In some embodiments the cell in the subject is a cancer cell. In some embodiments the cancer cell in the subject is a breast cancer cell. In some embodiments the cancer cell in the subject is a lung cancer cell. In some embodiments the cancer cell in the subject is a colorectal cancer cell. In some embodiments the contacting or introducing comprises administrating the compound to the subject. In some embodiments the subject is a mammal.

The presently-disclosed subject matter further includes a method of inhibiting cancer cell migration in a subject. In some embodiments, the method involves identifying the subject as having a cancer; and administering to the subject an effective amount of a compound, wherein the compound is selected from the group consisting of: amiloride, azelastine, bazedoxifene acetate, BIBW2992, DL-carnitine, L-carnitine, cyclosporin A, dopamine, gallic acid, gemcitabine, loperamide, manidipine, marimastat, methacycline, mubritinib, P1015, P1025, P1029, palbociclib, pexidartinib, rosiglitazone, tazemetostat, tebipenem pivoxil, teneligliptin, trospium chloride, and pharmaceutically-acceptable salts thereof. In some embodiments, the cancer cell is a breast cancer cell. In some embodiments, the cancer cell is a lung cancer cell. In some embodiments, the cancer cell is a colorectal cancer cell. In some embodiments, the subject is a mammal.

In some embodiments of inhibiting cancer cell migration, the compound is selected from the group consisting of: amiloride, azelastine, DL-carnitine, L-carnitine, gallic acid, loperamide, tebipenem pivoxil, and pharmaceutically-acceptable salts thereof. In some embodiments the compound is amiloride or a pharmaceutically-acceptable salt thereof. In some embodiments the compound is azelastine or a pharmaceutically-acceptable salt thereof. In some embodiments the compound is loperamide or a pharmaceutically-acceptable salt thereof.

In some embodiments of inhibiting cancer cell migration, the compound is provided in a pharmaceutical composition further comprising a pharmaceutically-acceptable carrier.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are used, and the accompanying drawings of which:

FIG. 1 illustrates the hydroxylation activities of purified recombinant PLOD2, including wild type (WT) PLOD2, which has hydroxylation activity, and a hydroxylation-deficient mutant type (MT) PLOD2, which lacks hydroxylation activity.

FIG. 2 includes the relative PLOD2 inhibitory activity of 1400 FDA-approved compounds screened in vitro at concentrations of 50 μM and 0.1 μM.

FIG. 3. includes the relative PLOD2 inhibitory activity of 25 FDA-approved compounds, which were capable of achieving such activity at a dose of 50 μM.

FIG. 4 includes a series of dose response curves for selected chemicals with 0.1 PLOD2.

FIG. 5 illustrates the inhibitory activity of the selected PLOD2 inhibitors (tebipenem pivoxil, DL-carnitine, L-carnitine, azelastine, amiloride, and gallic acid) on breast cancer progression in 3D culture. MDA-MB-231 cells were treated with the 20 μM chemicals in 3D culture for 3 days, and invasive growth was quantified under microscope.

FIG. 6. includes phase images and quantification data show that loperamide treatment (10 μM) inhibits invasive growth of MDA-MB-231 cells in 3D culture.

### DESCRIPTION OF EXEMPLARY EMBODIMENTS

The details of one or more embodiments of the presently-disclosed subject matter are set forth in this document. Modifications to embodiments described in this document, and other embodiments, will be evident to those of ordinary skill in the art after a study of the information provided in this document. The information provided in this document, and particularly the specific details of the described exemplary embodiments, is provided primarily for clearness of understanding and no unnecessary limitations are to be understood therefrom. In case of conflict, the specification of this document, including definitions, will control.

The presently-disclosed subject matter is based, in part, to the discovery that certain compounds previously-approved for unrelated indications by the FDA (“FDA-approved compounds”) possessed unexpected procollagen-lysine, 2-oxoglutarate 5-dioxygenases 2 (PLOD2) inhibitory activity. The presently-disclosed subject matter is also based, in part, to the discovery that certain FDA-approved compounds are capable of inhibiting migration or invasion of cancer cells. These identified compounds are set forth in Table 1.

TABLE 1

| Compound Name | Compound Structure |
|---|---|
| Amiloride | 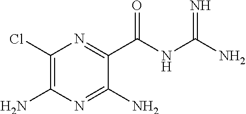 [SMILES: N=C(N)NC(=O)c1nc(Cl)c(N)nc1N] |
| Azelastine | 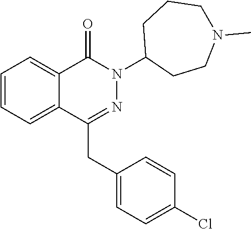 [SMILES: CN1CCCC(n2nc(Cc3ccc(Cl)cc3)c3ccccc3c2=O)CC1] |
| Bazedoxifene Acetate | 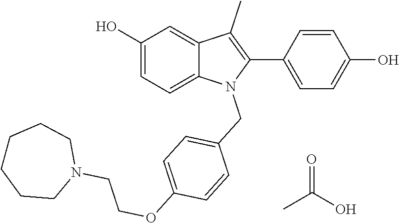 |
| BIBW2992 | 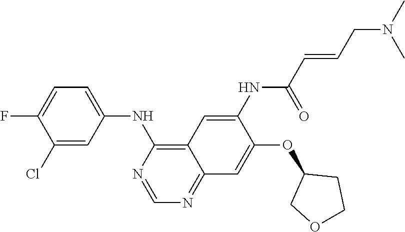 [SMILES: CN(C)C/C=C/C(=O)Nc1cc2c(Nc3ccc(F)c(Cl)c3)ncnc2cc1O[C@H]1CCOC1] |
| DL-Carnitine | 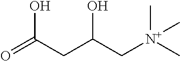 |
| L-Carnitine | 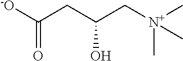 |
| Cyclosporin A | 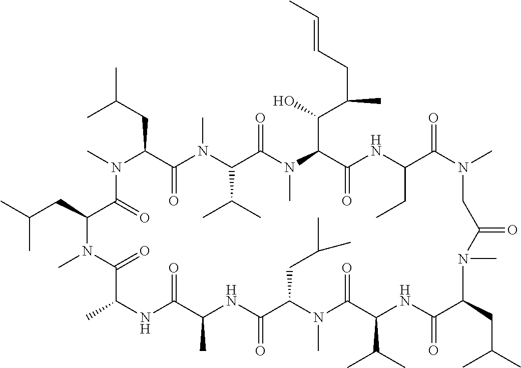 |
| Dopamine | 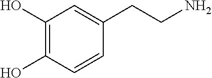 [SMILES: NCCc1ccc(O)c(O)c1] |
| Gallic acid | 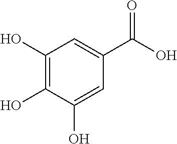 [SMILES: O=C(O)c1cc(O)c(O)c(O)c1] |
| Gemcitabine | 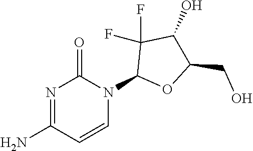 [SMILES: Nc1ccn([C@@H]2O[C@H](CO)[C@@H](O)C2(F)F)c(=O)n1] |
| Loperamide | 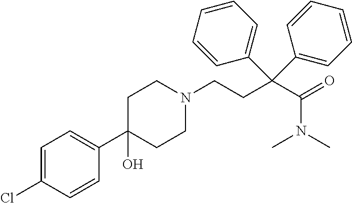 [SMILES: CN(C)C(=O)C(CCN1CCC(O)(c2ccc(Cl)cc2)CC1)(c1ccccc1)c1ccccc1] |
| Manidipine | 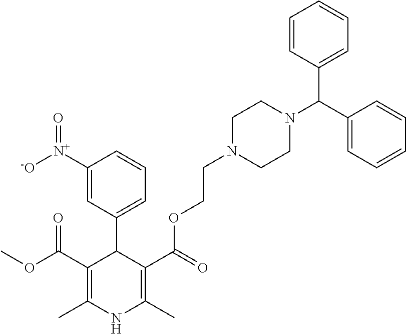 |
| Marimastat | 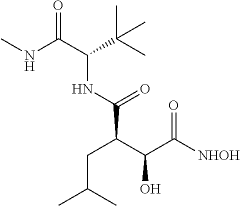 |
| Methacycline | 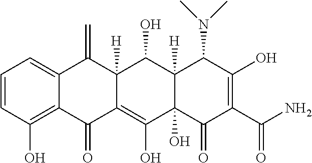 [SMILES: C=C1c2cccc(O)c2C(=O)C2=C(O)[C@]3(O)C(=O)C(C(N)=O)=C(O)[C@@H](N(C)C)[C@@H]3[C@@H](O)[C@H]12] |
| Mubritinib | 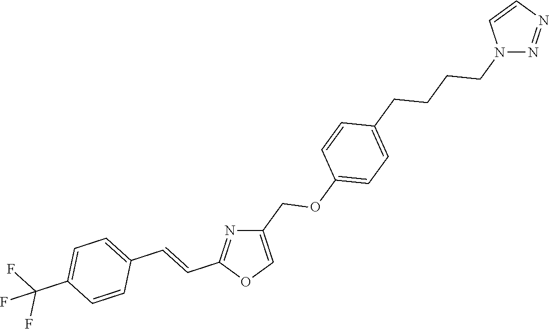 [SMILES: FC(F)(F)c1ccc(/C=C/c2nc(COc3ccc(CCCCn4ccnn4)cc3)co2)cc1] |
| P1015 | 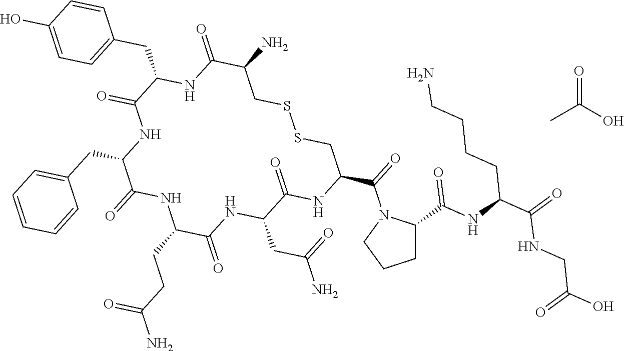 |
| P1025 | 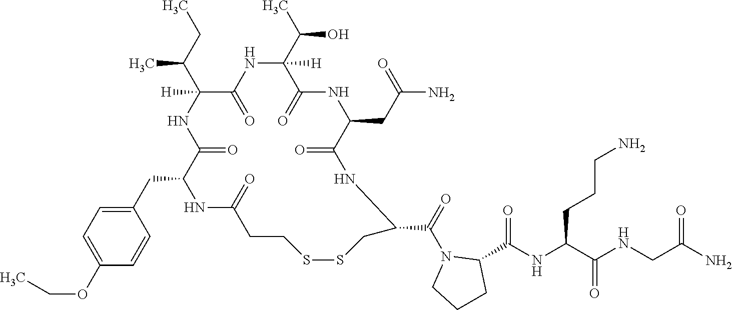 [SMILES: CCOc1ccc(C[C@H]2NC(=O)CCSSC[C@@H](C(=O)N3CCC[C@H]3C(=O)N[C@@H](CCCN)C(=O)NCC(N)=O)NC(=O)[C@H](CC(N)=O)NC(=O)[C@H]([C@@H](C)O)NC(=O)[C@H]([C@@H](C)CC)NC2=O)cc1] |
| P1029 | 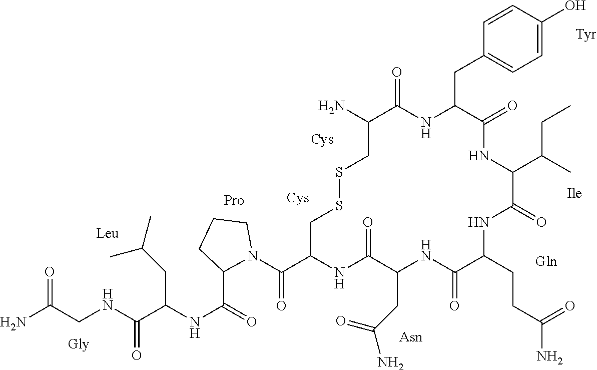 [SMILES: CCC(C)C1NC(=O)C(Cc2ccc(O)cc2)NC(=O)C(N)CSSCC(C(=O)N2CCCC2C(=O)NC(CC(C)C)C(=O)NCC(N)=O)NC(=O)C(CC(N)=O)NC(=O)C(CCC(N)=O)NC1=O] |
| Palbociclib | 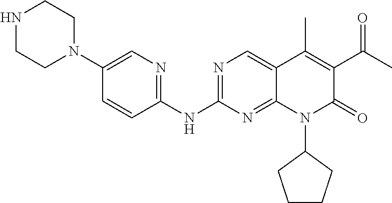 [SMILES: CC(=O)c1c(C)c2cnc(Nc3ccc(N4CCNCC4)cn3)nc2n(C2CCCC2)c1=O] |
| Pexidartinib | 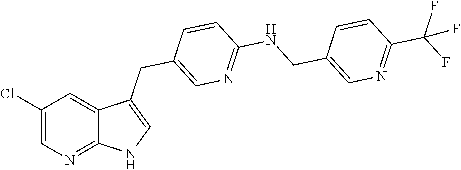 [SMILES: FC(F)(F)c1ccc(CNc2ccc(Cc3c[nH]c4ncc(Cl)cc34)cn2)cn1] |
| Rosiglitazone | 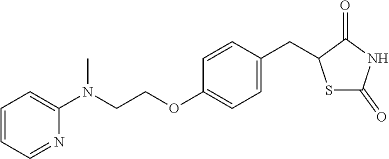 [SMILES: CN(CCOc1ccc(CC2SC(=O)NC2=O)cc1)c1ccccn1] |
| Tazemetostat | 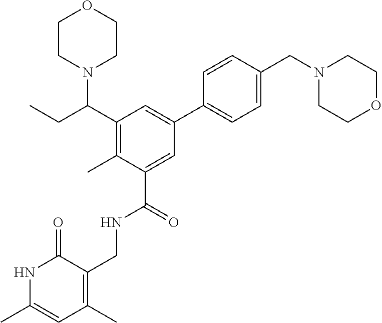 [SMILES: CCC(c1cc(-c2ccc(CN3CCOCC3)cc2)cc(C(=O)NCc2c(C)cc(C)[nH]c2=O)c1C)N1CCOCC1] |
| Tebipenem pivoxil | 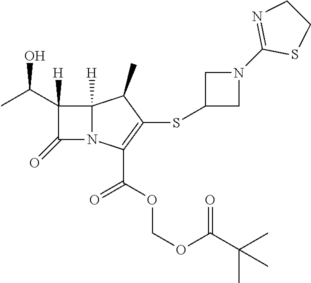 [SMILES: C[C@@H](O)[C@H]1C(=O)N2C(C(=O)OCOC(=O)C(C)(C)C)=C(SC3CN(C4=NCCS4)C3)[C@H](C)[C@H]12] |
| Teneligliptin | 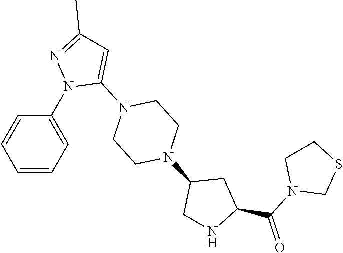 [SMILES: Cc1cc(N2CCN([C@@H]3CN[C@H](C(=O)N4CCSC4)C3)CC2)n(-c2ccccc2)n1] |
| Trospium chloride | 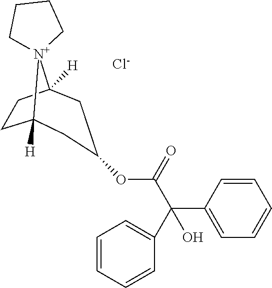 [SMILES: O=C(O[C@H]1C[C@H]2CC[C@@H](C1)[N+]21CCCC1)C(O)(c1ccccc1)c1ccccc1 ; [Cl-]] |

The indications and previously known utilities of these FDA-approved compounds are not related to PLOD2 activity or cancer treatment. Indeed, such previously-known indications were quite distinct. For example, amiloride is used as a diuretic in the context of hypertension, heart failure, and edema. For another example, azelastine is used to treat nasal symptoms, such as runny, itchy, or stuffy nose, sneezing, and post-nasal drip. For another example, loperamide is used in treating diarrhea. For yet another example, tebipenem pivoxil is used as an antibiotic.

The presently-disclosed subject matter includes a method of inhibiting the expression or activity of PLOD2 in a cell, which involves contacting the cell with or introducing into the cell an effective amount of a compound selected from the group consisting of: amiloride, azelastine, bazedoxifene acetate, BIBW2992, DL-carnitine, L-carnitine, cyclosporin A, dopamine, gallic acid, gemcitabine, loperamide, manidipine, marimastat, methacycline, mubritinib, P1015, P1025, P1029, palbociclib, pexidartinib, rosiglitazone, tazemetostat, tebipenem pivoxil, teneligliptin, trospium chloride, and pharmaceutically-acceptable salts thereof, wherein the contacting or introducing results in inhibition of expression or activity of the PLOD2 in the cell.

In some embodiments of the method of inhibiting the expression or activity of PLOD2 in a cell, the cell is cancer cell. In some embodiments, the cancer cell is a breast cancer cell. In some embodiments, the cancer cell is a lung cancer cell. In some embodiments, the cancer cell is a colorectal cancer cell.

In some embodiments of the method of inhibiting the expression or activity of PLOD2 in a cell, the cell is in a subject. In some embodiments of the method, the contacting or introducing comprises administrating the compound to the subject.

The presently-disclosed subject matter also includes a method of inhibiting cancer cell migration in a subject, which involves administering to the subject an effective amount of a compound, wherein the compound is selected from the group consisting of: amiloride, azelastine, bazedoxifene acetate, BIBW2992, DL-carnitine, L-carnitine, cyclosporin A, dopamine, gallic acid, gemcitabine, loperamide, manidipine, marimastat, methacycline, mubritinib, P1015, P1025, P1029, palbociclib, pexidartinib, rosiglitazone, tazemetostat, tebipenem pivoxil, teneligliptin, trospium chloride, and pharmaceutically-acceptable salts thereof.

In some embodiments, the method further involves identifying the subject as having a risk of cancer or having a cancer. In some embodiment, the subject is identified as having a risk of cancer because the subject was diagnosed with cancer in the past, which has a potential for recurrence. In some embodiments, the subject is identified as having cancer because the subject has been diagnosed with cancer. As used herein, the term “diagnosed” means having been subjected to an examination by a person of skill, for example, a physician, and found to have a condition that can be diagnosed or treated by the compounds, compositions, or methods disclosed herein. For example, “diagnosed with cancer” means having been subjected to an examination by a person of skill, for example, a physician, and found to have a condition that can be diagnosed or treated by a compound or composition that can impact, inhibit, or kill cancer cells.

In some embodiments of the method of inhibiting cancer cell migration in a subject, the cancer is selected from the group consisting of breast, lung, and colorectal cancer.

In some embodiments of the methods disclosed herein, the compound that is contacted, introduced, and/or administered can be provided in a in a pharmaceutical composition further comprising a pharmaceutically-acceptable carrier. As used herein, the term “pharmaceutically acceptable carrier” refers to sterile aqueous or nonaqueous solutions, dispersions, suspensions or emulsions, as well as sterile powders for reconstitution into sterile solutions or dispersions just prior to use.

As used herein, the term “effective amount” refers to an amount that is sufficient to achieve the desired result or to have an effect on an undesired condition. For example, a “therapeutically effective amount” refers to an amount that is sufficient to achieve the desired therapeutic result or to have an effect on undesired symptoms, but is generally insufficient to cause adverse side effects. The specific therapeutically effective dose level for any particular patient will depend upon a variety of factors including the disorder being treated and the severity of the disorder; the specific composition employed; the age, body weight, general health, sex and diet of the patient; the time of administration; the route of administration; the rate of excretion of the specific compound employed; the duration of the treatment; drugs used in combination or coincidental with the specific compound employed and like factors well known in the medical arts. For example, it is well within the skill of the art to start doses of a compound at levels lower than those required to achieve the desired therapeutic effect and to gradually increase the dosage until the desired effect is achieved. If desired, the effective daily dose can be divided into multiple doses for purposes of administration. Consequently, single dose compositions can contain such amounts or submultiples thereof to make up the daily dose. The dosage can be adjusted by the individual physician in the event of any contraindications. Dosage can vary, and can be administered in one or more dose administrations daily, for one or several days. Guidance can be found in the literature for appropriate dosages for given classes of pharmaceutical products. In further various aspects, a preparation can be administered in a “prophylactically effective amount”; that is, an amount effective for prevention of a disease or condition.

As used herein, the terms “administering” and “administration” refer to any method of providing a pharmaceutical preparation to a subject. Such methods are well known to those skilled in the art and include, but are not limited to, oral administration, transdermal administration, administration by inhalation, nasal administration, topical administration, intravaginal administration, ophthalmic administration, intraaural administration, intracerebral administration, rectal administration, and parenteral administration, including injectable such as intravenous administration, intra-arterial administration, intramuscular administration, and subcutaneous administration. Administration can be continuous or intermittent. In some embodiments, administration of a known compound can made by known routes for administering that compound. For example, in some embodiments, if tebipenem pivoxil is administered, it can be administered by an oral route, which is a known route for administration of tebipenem pivoxil.

As used herein, the terms “inhibit”, “inhibitor”, or “inhibiting” are not meant to require complete inhibition, but refers to a reduction in target activity. Such reduction can be a reduction of about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100%

As used herein, an “activity” of a polypeptide, such as an enzyme, refers to any activity exhibited by the polypeptide, such as catalyzing a particular biochemical reaction. Such activities can be empirically determined using methods known to those of ordinary skill in the art.

As used herein, “expression” refers to the process by which polypeptides are produced by transcription and translation of polynucleotides. The level of expression of a polypeptide can be assessed using any method known in art.

As used herein, the term “subject” refers to a human or animal subject. In some embodiments, the subject is a mammal. In some aspects, subject is a rodent. In other aspects of the invention, subject is a human.

As used herein, the term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

The present application can “comprise” (open ended) or “consist essentially of” the components of the present invention as well as other ingredients or elements described herein. As used herein, “comprising” is open ended and means the elements recited, or their equivalent in structure or function, plus any other element or elements which are not recited. The terms “having” and “including” are also to be construed as open ended unless the context suggests otherwise.

Following long-standing patent law convention, the terms “a”, “an”, and “the” refer to “one or more” when used in this application, including the claims. Thus, for example, reference to “a cell” includes a plurality of such cells, and so forth.

Unless otherwise indicated, all numbers expressing quantities of ingredients, properties such as reaction conditions, and so forth used in the specification and claims are to be understood as being modified in all instances by the term “about”. Accordingly, unless indicated to the contrary, the numerical parameters set forth in this specification and claims are approximations that can vary depending upon the desired properties sought to be obtained by the presently-disclosed subject matter.

As used herein, the term “about,” when referring to a value or to an amount of mass, weight, time, volume, concentration or percentage is meant to encompass variations of in some embodiments ±20%, in some embodiments ±10%, in some embodiments ±5%, in some embodiments ±1%, in some embodiments ±0.5%, in some embodiments ±0.1%, in some embodiments ±0.01%, and in some embodiments ±0.001% from the specified amount, as such variations are appropriate to perform the disclosed method.

As used herein, ranges can be expressed as from “about” one particular value, and/or to “about” another particular value. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

As used herein, “optional” or “optionally” means that the subsequently described event or circumstance does or does not occur and that the description includes instances where said event or circumstance occurs and instances where it does not. For example, an optionally variant portion means that the portion is variant or non-variant.

While the terms used herein are believed to be well understood by those of ordinary skill in the art, certain definitions are set forth to facilitate explanation of the presently-disclosed subject matter.

Unless defined otherwise, all technical and scientific terms used herein have the same meaning as is commonly understood by one of skill in the art to which the invention(s) belong.

All patents, patent applications, published applications and publications, GenBank sequences, databases, websites and other published materials referred to throughout the entire disclosure herein, unless noted otherwise, are incorporated by reference in their entirety.

Where reference is made to a URL or other such identifier or address, it understood that such identifiers can change and particular information on the internet can come and go, but equivalent information can be found by searching the internet. Reference thereto evidences the availability and public dissemination of such information.

As used herein, the abbreviations for any protective groups, amino acids and other compounds, are, unless indicated otherwise, in accord with their common usage, recognized abbreviations, or the IUPAC-IUB Commission on Biochemical Nomenclature (see, Biochem. (1972) 11(9):1726-1732).

Although any methods, devices, and materials similar or equivalent to those described herein can be used in the practice or testing of the presently-disclosed subject matter, representative methods, devices, and materials are described herein.

The presently-disclosed subject matter is further illustrated by the following specific but non-limiting examples. The following examples may include compilations of data that are representative of data gathered at various times during the course of development and experimentation related to the present invention.

### EXAMPLES

### Example 1: Expression and Purification of PLOD2 from Mammalian Cells

In prior activity measurement studies, PLOD2 had been expressed in E. Coli (Feldman 1996) and insect cells (van Biesen 1996). However, in the present study, PLOD2 was expressed in Eukaryotic cell 293 FT.

293FT cells were plated into 15 cm dishes for 24 hours before transfection. The expression plasmid pCDH PLOD2-flag was constructed and transfected into 293FT cells with Fugene following the protocol. Forty-eight hours after transfection, the transfected 293FT cells were harvested and lysed in a HGLB buffer.

The protein lysate was incubated with anti-flag gel at 4° C. overnight. Following incubation, unspecific binding protein was washed with NTE-2 buffer 6 times. The PLOD2 was eluted with 3*flag peptides.

### Example 2: Verification of PLOD2 Hydroxylation Activity

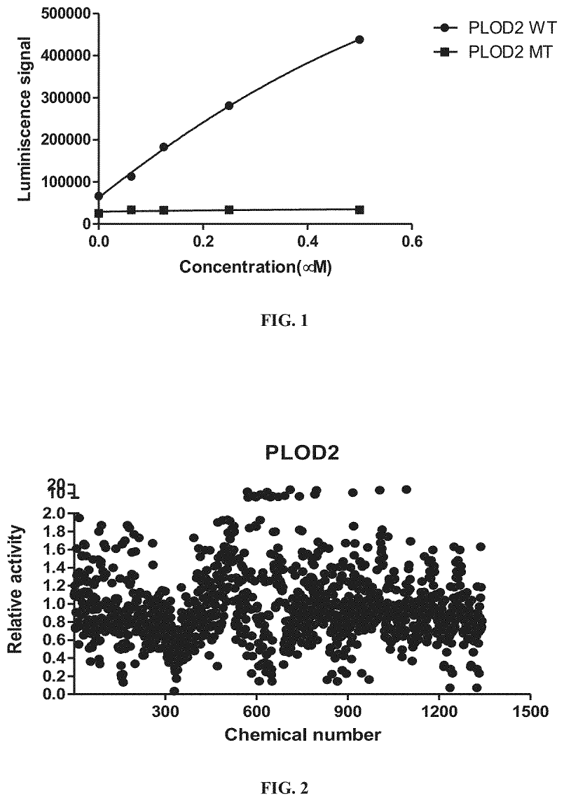

Purified PLOD2 was incubated with the substrate (IKG)₃ and αKG for approximately 1 hour. Succinate production was measured Succinate Glo™ JmjC Demethylase Assay Kit from Promega (Madison, Wis., cat. CS1747A04). With reference to FIG. 1, the results are presented as a function of concentration of wild type (WT) PLOD2 or hydroxylation-deficient mutant type (MT) PLOD2. As illustrated, succinate production was induced with the increased concentration of PLOD2 WT (black circles), while the PLOD2 MT (black squares) did not induce the succinate production. These data establish that the purified recombinant WT PLOD2 has hydroxylation activity.

### Example 3: Identification and Verification of PLOD2 Inhibitors

A High-Throughput Lysyl Hydroxylase (LH) Assay was developed to measure PLOD2 activity using the Succinate Glo™ Kit to identify PLOD2 inhibitors (Guo 2017; Devkota 2019). Using this method, a library of FDA-approved drugs was screened to identify compounds having potential utility as PLOD2 inhibitors. More than 1400 FDA-approved compounds were screened.

PLOD2 (0.1 μM) was incubated with 50 μM compounds at 4° C. for 2 h. Following the incubation, other reaction factors were added to make the reaction mixture (50 μM FeSO₄, 100 μM AKG, 500 μM ascorbate, 1.5 μM calalase, 1 mM (IKG)₃ and 50 mM Hepes). The reaction was incubated at 37° C. for 1 h before adding reagent 1 to the reaction solution. After 1 additional hour, reagent II was added to the reaction solution for 10 min. Luminescence was measured to determine relative activity of PLOD2 in the reaction mixture, with a higher value being associated with a higher activity.

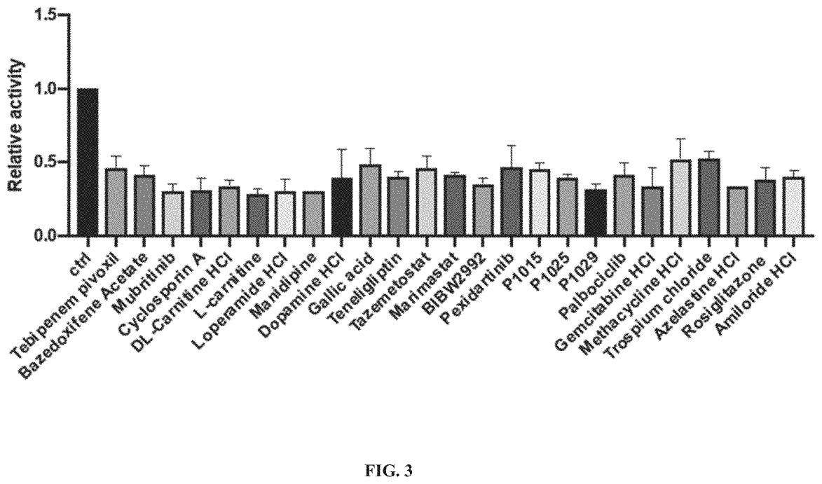

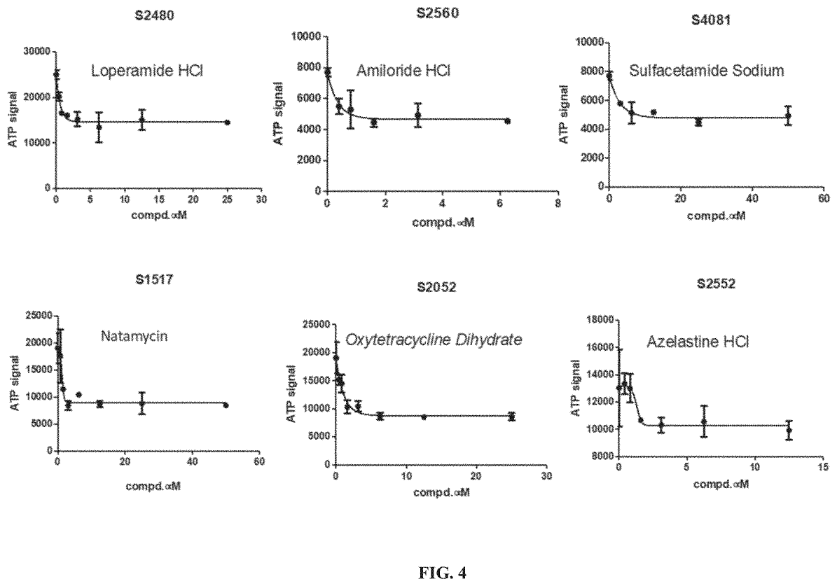

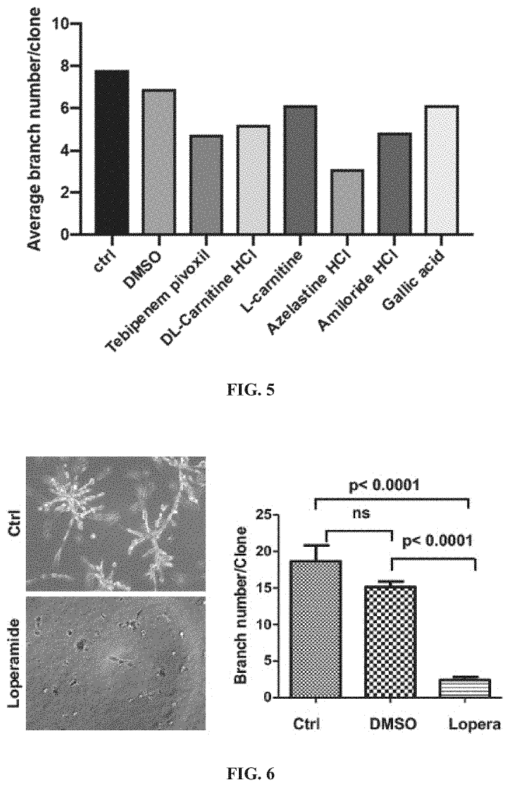

With reference to FIG. 2, the relative in vitro PLOD2 inhibitory activity of compounds was determined at concentrations of 50 μM and 0.1 μM. Twenty-five compounds were identified as having PLOD2 inhibitory activity at 50 as presented in the bar graph of FIG. 3. From these compounds six (6) were selected for additional testing, including assessment. FIG. 4 includes a series of dose response curves, illustrating the inhibitory activity over a range of doses. The twenty-five (25) identified inhibitors are presented in Table 1, and were not previously known to have any PLOD2 inhibitor activity.

### Example 4: Attenuation of Cancer Cell Invasion

PLOD2 upregulation is closely related with cancer progression and invasion. (Gilkes 2013b; Chen 2015; Eisinger-Mathason 2013; Monferrer 2019; Guo 2018; Chen 2016; Kiyozumi 2018; Okumura 2018; Xu 2017).

3D culture assay is an established platform to study cancer cell invasion, and the ability of compounds to attenuate such cell invasion (Zhang 2015). To verify whether the identified PLOD2 inhibitors have the ability attenuating cancer cell invasion, MDA-MB-231 cells were treated with the identified inhibitors (test compounds) in 3D culture. The MDA-MB-231 cell line is an epithelial, human breast cancer cell line.

Briefly, to conduct the 3D culture assay, 12-well plates were coated with 1204, matrigel per well. After incubating the plates at 37° C. for 15 minutes, MDA-MB-231 cells were plated with about 300 cells per well. The plates were allowed to stand for 45 min in the 37° C. incubator to let the cells deposit into the matrigel. The cells were then covered with 10% matrigel, which contained test compounds. The final concentration of each test compound was 20 μM. After culturing for 48 hours, pictures were taken and quantified using GraphPrism 5.

With reference to FIG. 6 and FIG. 7, the tested compounds were found to inhibit invasive growth or tumor progression in 3D culture. These results demonstrate that identified PLOD2 inhibitors can inhibit cancer cell invasion or migration.

All publications, patents, and patent applications mentioned in this specification are herein incorporated by reference to the same extent as if each individual publication, patent, or patent application was specifically and individually indicated to be incorporated by reference, including the references set forth in the following list:

### REFERENCES

Albaugh V. L., et al. (2017). Proline precursors and collagen synthesis: biochemical challenges of nutrient supplementation and wound healing. J. Nutr. 147, 2011-2017. 10.3945/jn.117.256404
Barnes M. J., et al. (1974). Age-related variations in hydroxylation of lysine and proline in collagen. Biochem. J. 139, 461-468. 10.1042/bj1390461
Chen Y., et al. (2015). Lysyl hydroxylase 2 induces a collagen cross-link switch in tumor stroma. J. Clin. Invest. 125, 1147-1162. 10.1172/JCI74725.
Chen Y., et al. (2016). Lysyl hydroxylase 2 is secreted by tumor cells and can modify collagen in the extracellular space. J. Biol. Chem. 291, 25799-25808. 10.1074/jbc.M116.759803.
Cheon, D.-J., Tong, Y., Sim, M.-S., Dering, J., Berel, D., Cui, X., et al. (2014). A collagen-remodeling gene signature regulated by tgf-β signaling is associated with metastasis and poor survival in serous ovarian cancer. Clin. Cancer Res. 20. 711-723. doi: 10.1158/1078-0432.CCR-13-1256.
Devkota A. K., et al. (2019) Development of a High-Throughput Lysyl Hydroxylase (LH) Assay and Identification of Small-Molecule Inhibitors against LH2. SLAS Discov. 24(4):484-491. doi: 10.1177/2472555218817057.
Egeblad, M., et al. (2010). Dynamic interplay between the collagen scaffold and tumor evolution. Curr. Opin. Cell Biol. 22, 697-706. doi: 10.1016/j.ceb.2010.08.015
Eisinger-Mathason T. S. K., et al. (2013). Hypoxia-dependent modification of collagen networks promotes sarcoma metastasis. Cancer Discov. 3, 1190-1205. 10.1158/2159-8290.CD-13-0118.
Feldman S R, Fleischer A B Jr, Reboussin D M, Rapp S R, Exum M L, Clark A R, Nurre L. The self-administered psoriasis area and severity index is valid and reliable. J Invest Dermatol. 1996 January; 106(1):183-6. doi: 10.1111/1523-1747.ep12329912.
Gilkes D. M., et al. (2013). Hypoxia-inducible Factor 1 (HIF-1) Promotes Extracellular Matrix Remodeling under Hypoxic Conditions by Inducing P4HA1, P4HA2, and PLOD2 Expression in Fibroblasts. J. Biol. Chem. 278, 40967-40972.
Gilkes D. M., et al. (2013b). Procollagen lysyl hydroxylase 2 is essential for hypoxia-induced breast cancer metastasis. Mol. Cancer Res. 11, 456-466. 10.1158/1541-7786.MCR-12-0629.
Gjaltema R. A. and Bank R A. (2017) Molecular insights into prolyl and lysyl hydroxylation of fibrillar collagens in health and disease. Crit Rev Biochem Mol Biol. 52(1):74-95. doi: 10.1080/10409238.2016.1269716.
Guo H-F., et al. (2017) A scalable lysyl hydroxylase 2 expression system and luciferase-based enzymatic activity assay. Arch. Biochem. Biophys, 618, 45-51.
Guo H-F., et al. (2018) Pro-metastatic collagen lysyl hydroxylase dimer assemblies stabilized by Fe2+-binding. Nat Commun 9(1): 512.
Hausmann E. (1967). Cofactor requirements for the enzymatic hydroxylation of lysine in a polypeptide precursor of collagen. Biochim. Biophys. Acta 133, 591-593. 10.1016/0005-2795(67)90566-1.
Kivirikko Ki P. T. (1998). Collagen hydroxylases and the protein disulfide isomerase subunit of prolyl 4-hydroxylases. Adv. Enzymol. Relat. Areas Mol. Biol. 72, 325-398. 10.1002/9780470123188.ch9
Kiyozumi, Y. (2018) PLOD2 as a potential regulator of peritoneal dissemination in gastric cancer. Int. J. Cancer 143(5): 1202-1211.
Li S. S., et al. (2020) Overexpressing PLOD family genes predict poor prognosis in gastric cancer. J Cancer. 11(1):121-131. doi: 10.7150/jca.35763.
Monferrer E., et al. (2019) High Oct4 expression: implications in the pathogenesis of neuroblastic tumours. BMC Cancer 2019 Aug. 27; 19(1).
Okumura Y. et al. (2018) Hypoxia-Induced PLOD2 is a Key Regulator in Epithelial-Mesenchymal Transition and Chemoresistance in Biliary Tract Cancer. Ann. Surg. Oncol. 25(12): 3728-3737.
Oudin, M. J.; Jonas, O., Kosciuk, T., Broye; L. C.; Guido, B. C., Wyckoff; J., et al. (2016). Tumor cell-driven extracellular matrix remodeling drives haptotaxis during metastatic progression. Cancer Discov, 6, 516-531. doi: 10.1158/2159-8290.CD-15-1183.
Dollard, J. W. (2004). Tumour-educated macrophages promote tumour progression and metastasis. Nat. Rev. Cancer 4, 71-78. doi: 10.1038/nrc1256.
Qi Y, Xu R. Roles of PLODs in Collagen Synthesis and Cancer Progression. Front Cell Dev Biol. 2018; 6:66. Published 2018 Jun. 28. doi:10.3389/fcell.2018.00066
Rautavuoma K., et al. (2004). Premature aggregation of type IV collagen and early lethality in lysyl hydroxylase 3 null mice. Proc. Natl. Acad. Sci. U.S.A. 101, 14120-14125. 10.1073/pnas.0404966101.
Rhoads R. E. and Udenfriend S. (1968). Decarboxylation of alpha-ketoglutarate coupled to collagen proline hydroxylase. Proc. Natl. Acad. Sci. U.S.A. 60, 1473-1478. 10.1073/pnas.60.4.1473.
Simon, M. S. and T. S. K. Eisinger, U.S. Patent Application Publication No. 2014/0356287 for PLOD2 as a Target of Intervention for Sarcoma Metastasis.
Sun, L., Wang, D., Li, X., Zhang, L., Zhang, H., and Zhang, Y. (2016). Extracellular matrix protein ITGBL1 promotes ovarian cancer cell migration and adhesion through Wnt/PCP signaling and FAK/SRC pathway. Biomed. Pharmacother, 81, 145-151. doi: 10.1016/j.biopha.2016.03.053.
Takaluoma K., et al. (2007) Lysyl hydroxylase 2 is a specific telopeptide hydroxylase, while all three isoenzymes hydroxylate collagenous sequences. Matrix Biol. 2007 June; 26(5):396-403. doi: 10.1016/j.matbio.2007.01.002. Epub 2007 Jan. 16. PMID: 17289364.
Uzawa K., et al. (1999) Differential expression of human lysyl hydroxylase genes, lysine hydroxylation, and cross-linking of type I collagen during osteoblastic differentiation in vitro. J. Bone Miner. Res. 14: 1272-1280.
Valtavaara M., et al. (1998). Primary structure, tissue distribution, and chromosomal localization of a novel isoform of Lysyl Hydroxylase (Lysyl Hydroxylase 3). J. Biol. Chem. 273, 12881-12886. 10.1074/jbc.273.21.12881.
Van Biesen (1996) Go-protein α-Subunits Activate. Mitogen-activated Protein Kinase via a Novel Protein Kinase C-dependent Mechanism. J Biol Chem. 1996 Apr. 19; 271(16):9398-402.
Xiong, G., et al. (2014). Prolyl-4-hydroxylase a subunit 2 promotes breast cancer progression and metastasis by regulating collagen deposition. BMC Cancer 14:1. doi: 10.1186/1471-2407-14-1
Xiong, G.-F., and Xu, R. (2016). Function of cancer cell-derived extracellular matrix in tumor progression. J. Cancer Metastas. Treat 2, 3.57-364. doi: 10.20517/2394-4722.2016.08
Xu Y., et al. (2017) Procollagen-lysine 2-oxoglutarate 5-dioxygenase 2 promotes hypoxia-induced glioma migration and invasion. Oncotarget. 2017; 8(14):23401-23413. doi:10.18632/oncotarget.15581
Zhang Q. Y., et al. (2015) Autophagy-mediated HMGB1 release promotes gastric cancer cell survival via RAGE activation of extracellular signal-regulated kinases 1/2. Oncol Rep. 2015 April; 33(4):1630-8. doi: 10.3892/or.2015.3782. Epub 2015 Feb. 4. PMID: 25652880; PMCID: PMC4358082.

It will be understood that various details of the presently disclosed subject matter can be changed without departing from the scope of the subject matter disclosed herein. Furthermore, the foregoing description is for the purpose of illustration only, and not for the purpose of limitation.

What is claimed is:

1. A method of inhibiting expression or activity of procollagen-lysine, 2-oxoglutarate 5-dioxygenases 2 (PLOD2) in a cell comprising contacting the cell with or introducing into the cell an effective amount of a compound selected from the group consisting of: amiloride, azelastine, bazedoxifene acetate, BIBW2992, DL-carnitine, L-carnitine, cyclosporin A, dopamine, gallic acid, gemcitabine, loperamide, manidipine, marimastat, methacycline, mubritinib, P1015, P1025, P1029, palbociclib, pexidartinib, rosiglitazone, tazemetostat, tebipenem pivoxil, teneligliptin, trospium chloride, and pharmaceutically-acceptable salts thereof, wherein the contacting or introducing results in inhibition of expression or activity of the PLOD2 in the cell.

2. The method of claim 1, wherein the compound is selected from the group consisting of: amiloride, azelastine, DL-carnitine, L-carnitine, gallic acid, loperamide, tebipenem pivoxil, and pharmaceutically-acceptable salts thereof.

3. The method of claim 1, wherein the compound is amiloride or a pharmaceutically-acceptable salt thereof.

4. The method of claim 1, wherein the compound is azelastine or a pharmaceutically-acceptable salt thereof.

5. The method of claim 1, wherein the compound is loperamide or a pharmaceutically-acceptable salt thereof.

6. The method of claim 1, wherein the compound is provided in a pharmaceutical composition further comprising a pharmaceutically-acceptable carrier.

7. The method of claim 1, wherein the cell is cancer cell.

8. The method of claim 7, wherein the cancer cell is a breast, lung, or colorectal cancer cell.

9. The method of claim 1, wherein the cell is in a subject.

10. The method of claim 9, wherein the cell is a cancer cell.

11. The method of claim 10, wherein the cancer cell is a breast, lung, or colorectal cancer cell.

12. The method of claim 9, wherein the contacting or introducing comprises administrating the compound to the subject.

13. The method of claim 12, wherein the subject is a mammal.

14. A method of inhibiting cancer cell migration in a subject, comprising: identifying the subject as having a cancer or a risk thereof; and administering to the subject an effective amount of a compound selected from the group consisting of: amiloride, azelastine, bazedoxifene acetate, BIBW2992, DL-carnitine, L-carnitine, cyclosporin A, dopamine, gallic acid, gemcitabine, loperamide, manidipine, marimastat, methacycline, mubritinib, P1015, P1025, P1029, palbociclib, pexidartinib, rosiglitazone, tazemetostat, tebipenem pivoxil, teneligliptin, trospium chloride, and pharmaceutically-acceptable salts thereof.

15. The method of claim 14, wherein the cancer is breast, lung, or colorectal cancer.

16. The method of claim 14, wherein the compound is selected from the group consisting of: amiloride, azelastine, DL-carnitine, L-carnitine, gallic acid, loperamide, tebipenem pivoxil, and pharmaceutically-acceptable salts thereof.

17. The method of claim 14, wherein the compound is amiloride or a pharmaceutically-acceptable salt thereof.

18. The method of claim 14, wherein the compound is azelastine or a pharmaceutically-acceptable salt thereof.

19. The method of claim 14, wherein the compound is loperamide or a pharmaceutically-acceptable salt thereof.

20. The method of claim 14, wherein the compound is provided in a pharmaceutical composition further comprising a pharmaceutically-acceptable carrier.
